(19) United States
(12) Patent Application Publication
(10) Pub. No.: US 2011/0125208 A1
(43) Pub. Date: May 26, 2011

(54) METHODS AND SYSTEMS TO MONITOR CARDIAC CONTRACTILITY

(76) Inventors: Edward Karst, S.Pasadena, CA (US); Brian Jeffrey Wenzel, San Jose, CA (US); Allen J. Keel, San Francisco, CA (US); Wenbo Hou, Lancaster, CA (US); Taraneh Ghaffari Farazi, San Jose, CA (US)

(21) Appl. No.: 12/649,632

(22) Filed: Dec. 30, 2009

Related U.S. Application Data
(60) Provisional application No. 61/263,254, filed on Nov. 20, 2009.

(51) Int. Cl. A61N 1/362; A61B 5/02

(52) U.S. Cl. 607/17; 600/508

(57) ABSTRACT

An implanted sensor produces a signal that is indicative of changes in arterial blood volume, such as a photoplethysmography signal or an impedance plethysmography signal. A metric is determined from the signal for each of the plurality of periods. Changes in cardiac contractility are monitored based on changes in the determined metric.

### FIELD OF THE INVENTION

This application claims priority under 35 U.S.C. §119(e) to U.S. Provisional Patent Application No. 61/263,254, filed Nov. 20, 2009, entitled METHODS AND SYSTEMS TO MONITOR CARDIAC CONTRACTILITY, which is incorporated herein by reference.

### FIELD OF THE INVENTION

Embodiments of the present invention relate to implantable systems and methods for use therewith, for monitoring cardiac contractility.

### BACKGROUND OF THE INVENTION

Cardiac contractility, also referred to as myocardial contractility, is a term used in physiology to describe the performance of cardiac muscle.

Contractility is often defined as the intrinsic ability of a cardiac muscle fiber to contract at a given fiber length. Changes in the ability to produce force during contraction results from different degrees of binding between myosin (thick) and actin (thin) filaments. The degree of binding that occurs depends on concentration of calcium ions in the cell. In an intact heart, it is usually the action of the sympathetic nervous system (through catecholamines) which determines the concentration of calcium ions in the cytosol of cardiac muscle cells. Factors that cause an increase in contractility typically work by causing an increase in intracellular [Ca⁺⁺] during contraction.

Under one existing model, the factors of myocardial performance are considered to be heart rate, conduction velocity, preload, afterload and contractility. By this model, if myocardial performance changes while preload, afterload, heart rate and conduction velocity are all held constant, then the change in performance must be due to a change in contractility.

Contractility is an indicator of the strength of the heart, particularly the left ventricle, which must deliver the force that propels blood into the systemic arteries and throughout the body. It is often quantified by recording a left ventricular (LV) pressure waveform. The maximum rate of increase of the first derivative of LV pressure, or LV dP/dt max, provides an indication of contractility.

LV dP/dt max often occurs during isovolumic contraction, before the aortic valve has opened. However, the pressure generated by the heart's contractility quickly rises beyond diastolic blood pressure, resulting in a force of blood expulsion into the aorta. The major vessels distend rapidly and help to propagate the pressure wave through the arteries. The rate of increase in pressure with the arriving pressure wave contains information about the force that produced the wave and the compliance of the tubes that absorb and transmit the force.

Low cardiac contractility may be indicative of insufficient pump function or dyssynchrony in the heart's contraction.

### SUMMARY OF THE INVENTION

Embodiments of the present invention are related to implantable systems and methods for use therewith. Specific embodiments of the present invention relate to implantable systems that include an implantable sensor and methods for use therewith.

Embodiments of the present invention include using an implanted sensor to produce a signal that is indicative of changes in arterial blood volume. The implanted sensor can be an implanted photoplethysmography sensor to produce a photoplethysmography (PPG) signal, an implanted impedance plethysmography sensor to produce an impedance plethysmography (IPG) signal, or some other type of sensor.

Features of the signal indicative of changes in arterial blood volume can be used to determine cardiac contractility. Maximum increase in left ventricular pressure dP/dt during contraction is commonly regarded as an indicator of cardiac contractility. Absent any acute vascular remodeling, short-term changes in the force of the systolic pressure wave that propagates into the aorta and the arterial tree are positively correlated with cardiac contractility. Other features and feature combinations, such as a pulse waveform's FWHM (full-width, half max) and the time from maximum slope during systolic arterial distention to the dicrotic notch of the signal are also correlated to LV dP/dt max.

A metric can be determined from the signal for each of a plurality of periods. Changes in cardiac contractility can be monitored based on changes in the determined metric.

In one embodiment, the determined metric is indicative of an upward slope of the signal. For example, this can be a maximum upward slope, average upward slope or some other upward slope metric. An increase in the upward slope metric is indicative of an increase in the cardiac contractility. A decrease in the upward slope metric is indicative of a decrease in the cardiac contractility. No change in the upward slope metric is indicative of no change in the cardiac contractility.

In one embodiment, the determined metric is indicative of a time between two features of a cycle of the signal indicative of changes in arterial blood volume. The two features can occur between and inclusive of a foot (indicative of beginning of systole) and a dicrotic notch of the signal (indicative of the closing of the aortic valve during a cycle of the signal). The time can be a time from the foot to the dicrotic notch, a time from the maximum upward slope to the dicrotic notch, a time between two features of the upward slope, a time between the maximum upward slope and maximum downward slope, or some other time metric. A decrease in the time metric is indicative of an increase in the cardiac contractility. An increase in the time metric is indicative of a decrease of the cardiac contractility. No change in the time metric is indicative of no change in the cardiac contractility.

In one embodiment, the determined metric can be indicative of relative amplitude of the signal where the dicrotic notch occurs. An increase in this metric is indicative of an increase in the cardiac contractility. A decrease in the metric is indicative of a decrease in the cardiac contractility. No change in the metric is indicative of no change in the cardiac contractility.

The metric can also be determined from a derivative of the signal. For example, the first and second derivative of the signal can be used to obtain information about the cardiac contractility using zero crossings and other features.

At least one pacing parameter can be adjusted based on the monitored cardiac contractility. For example, the at least one pacing parameter can be adjusted to modify the monitored cardiac contractility be closer to a desired cardiac contractility.

A change in pacing parameters can be used to produce a change in monitored cardiac contractility. This change in monitored cardiac contractility can be used to determine the presence of dyssynchrony or a change in dyssynchrony.

A lack of capture by an implanted cardiac stimulation device can be determined based on the monitored cardiac contractility. In one embodiment, the lack of capture can be determined if the cardiac contractility is the same during pacing as it is during intrinsic activity, rather than the cardiac contractility changing to values typical for normal pacing with capture.

This description is not intended to be a complete description of, or limit the scope of, the invention. Other features, aspects, and objects of the various embodiments of the present invention can be obtained from a review of the specification, the figures, and the claims.

### BRIEF DESCRIPTION OF THE FIGURES

FIG. 1 is a high level flow diagram that is used to explain details for monitoring cardiac contractility with certain embodiments of the present invention.

FIG. 2 is a high level flow diagram of one embodiment of the present invention illustrating the modification of pacing parameters.

FIG. 3 is a high level flow diagram of an embodiment of the present invention in which changes in cardiac contractility are monitored along with the heart rate.

FIGS. 4A-4B includes exemplary signal waveforms that are used to show various slope related metrics that are indicative of cardiac contractility.

FIGS. 5A-5B includes exemplary signal waveforms that are used to show various time related metrics that are indicative of the cardiac contractility.

FIGS. 6A-6B includes exemplary signal waveforms that are used to show a dicrotic notch amplitude related metric that is indicative of the cardiac contractility.

FIG. 7A illustrates an exemplary implantable stimulation device that can be used to perform various embodiments of the present invention.

FIGS. 7B-7C illustrates exemplary implantable monitoring devices that can be used to perform various embodiments of the present invention.

FIG. 8 is a simplified block diagram that illustrates possible components of the implantable devices as shown in FIGS. 7A-7C.

### DETAILED DESCRIPTION OF THE INVENTION

The following description is of the best modes presently contemplated for practicing various embodiments of the present invention. The description is not to be taken in a limiting sense but is made merely for the purpose of describing the general principles of the invention. The scope of the invention should be ascertained with reference to the claims. In the description of the invention that follows, like numerals or reference designators will be used to refer to like steps, parts or elements throughout. In addition, the first digit of a reference number identifies the drawing in which the reference number first appears.

It would be apparent to one of skill in the art reading this description that the various embodiments of the present invention, as described below, may be implemented in many different embodiments of hardware, software, firmware, and/or the entities illustrated in the Figures. Any actual software, firmware and/or hardware described herein is not limiting of the present invention. Thus, the operation and behavior of the embodiments of the present invention will be described with the understanding that modifications and variations of the embodiments are possible, given the level of detail presented herein.

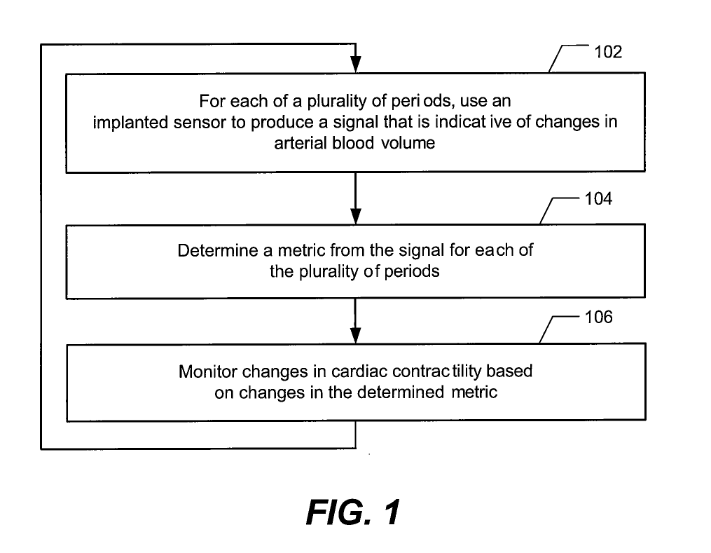

Various embodiments of the present invention for monitoring cardiac contractility will now be summarized beginning with a description of the high level flow diagram of FIG. 1. Where embodiments of the present invention are summarized with reference to the high level flow diagrams, various algorithmic steps are summarized in individual ‘blocks’. Such blocks describe specific actions or decisions that are made or carried out as the algorithm proceeds. Where a microcontroller (or equivalent) is employed, the flow diagrams presented herein provides the basis for a ‘control program’ that may be used by such a microcontroller (or equivalent) to effectuate the desired control of the implantable system. Those skilled in the art may readily write such a control program based on the flow diagram and other description presented herein. Embodiments of the present invention are not limited to the exact order and/or boundaries of the steps shown in the flow diagrams. In fact, many of the steps can be performed in a different order than shown, and many steps can be combined, or separated into multiple steps. All such variations are encompassed by the present invention. The only time order is important is where a step acts on the result of a previous step.

In step 102, for each of a plurality of periods, an implanted sensor is used to produce a signal that is indicative of changes in arterial blood volume. The sensor can be an implantable photoplethysmography (PPG) sensor to produce a photoplethysmography (PPG) signal, an implantable impedance plethysmography (IPG) sensor to produce an impedance plethysmography (IPG) signal, or some other type of sensor.

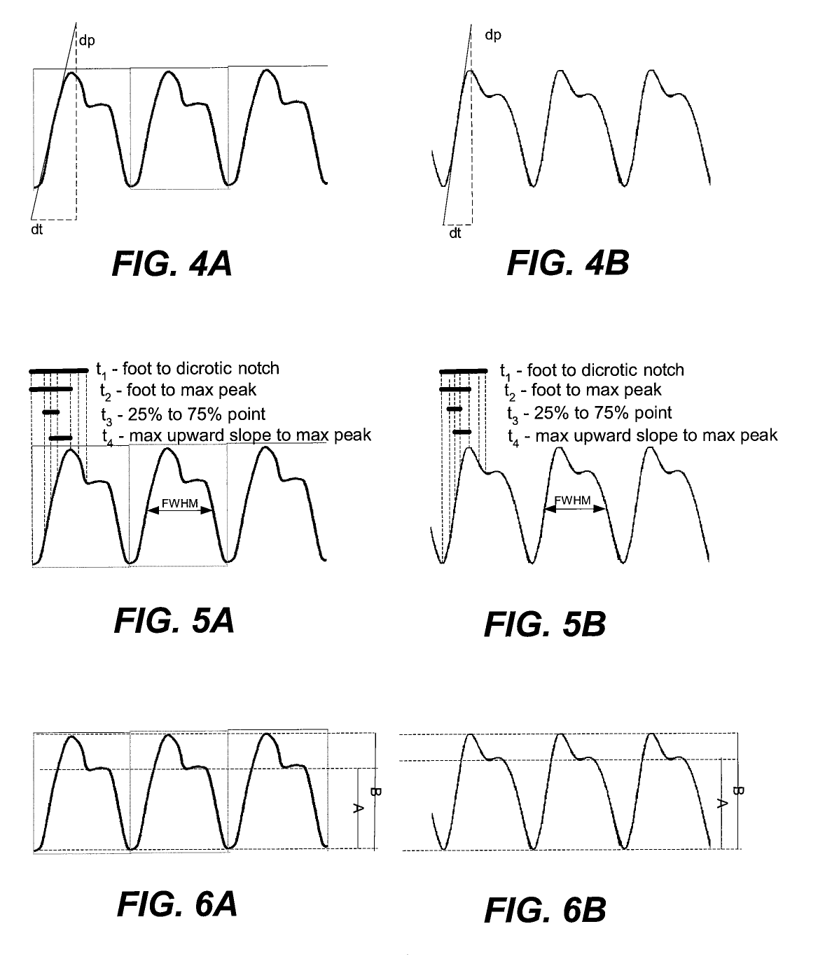

In step 104, for each of the plurality of periods, a metric is determined from the signal indicative of changes of arterial blood pressure. Exemplary metrics include upward slope metric, a metric related to time between two features of a cycle and an amplitude metric. Such metrics can be determined by analyzing the signal indicative of arterial blood volume. Additional details of such metrics are discussed below with reference to the FIGS. 4-6.

In step 106, changes in cardiac contractility are monitored based on changes in the determined metric. Changes in the metrics can be determined by comparing the metrics from different signal cycle periods.

Steps 102, 104 and 106 can be repeated from time to time, e.g., periodically, or in response to a triggering event. For example, steps 102, 104 and 106 can be performed substantially continually, or periodically (e.g., once an hour, a day, a week, or the like). Additionally, steps 102, 104 and 106 can be performed a periodically, e.g., in response to a triggering event

In an embodiment of the present invention, the signal indicative of changes in arterial blood volume can be a photoplethysmography signal. Volume changes in blood vessels occur in a pulsatile manner with each beat of the heart as blood flows in and out of a portion of the body. A PPG sensor produces waveform measurements reflecting changes in arterial blood volume. These waveforms measurements are similar to arterial pressure waveform measurements because changes in arterial pressure correspond to relative changes in arterial blood volume. For certain embodiments it is preferred that a plurality of cardiac cycles of the obtained PPG signal are averaged to produce a PPG waveform that is an averaged PPG waveform.

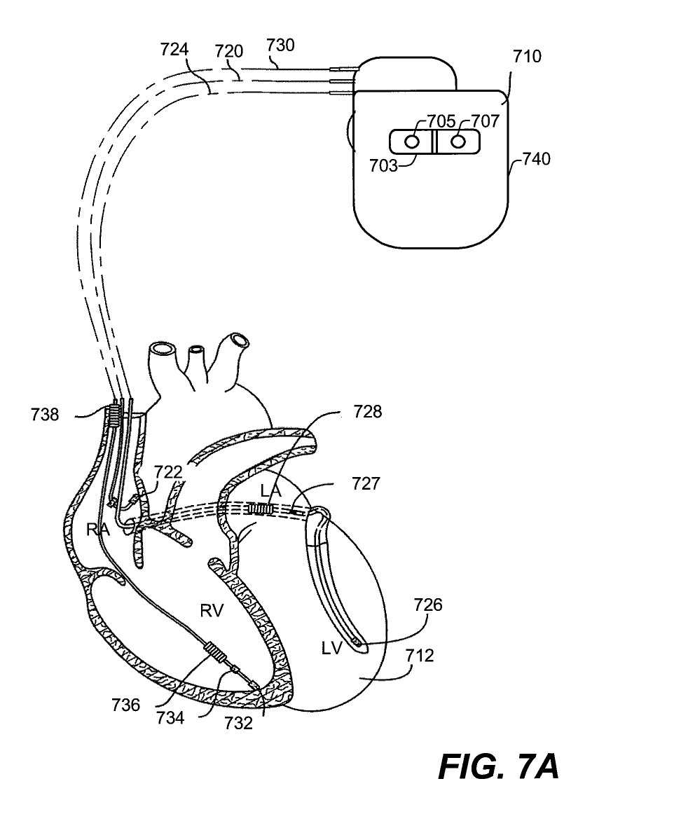

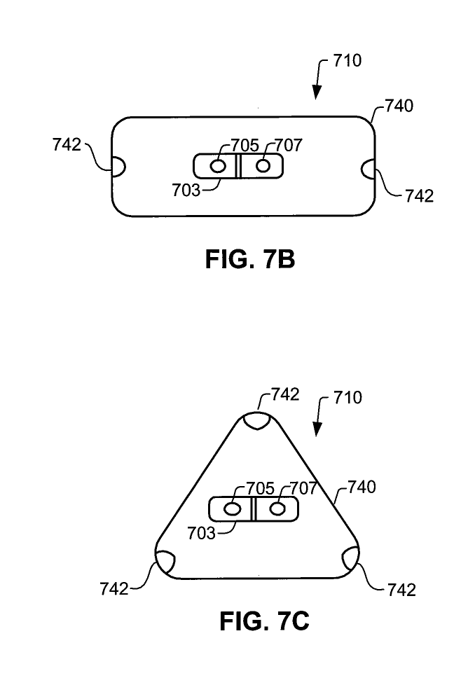

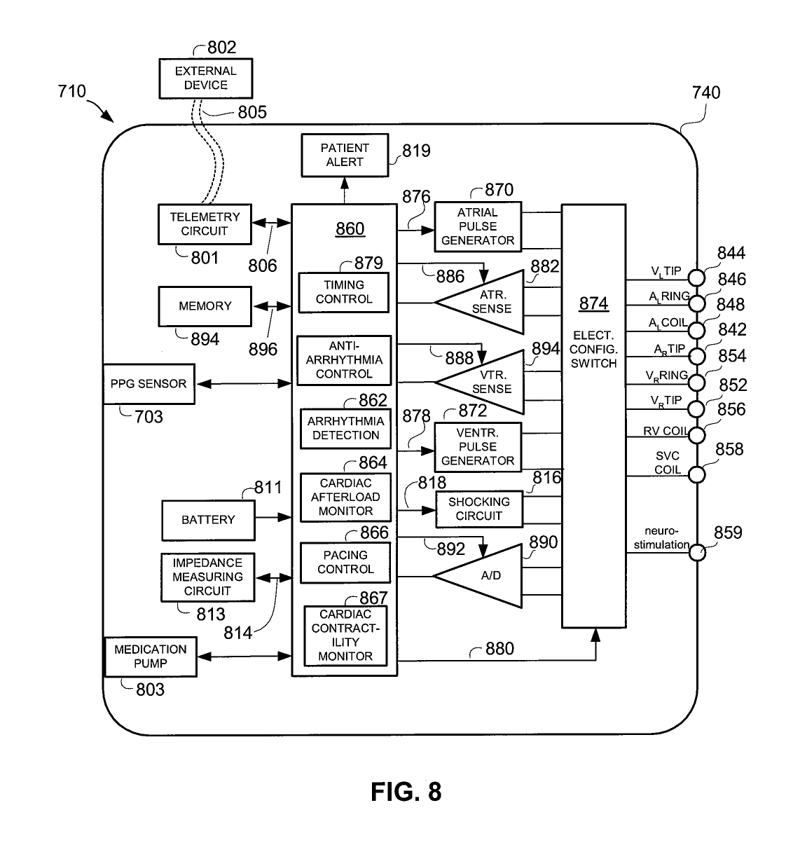

Exemplary PPG sensors are discussed below with reference to FIGS. 7A-7C. The PPG sensor can be implanted, e.g., in the pectoral region of a patient. Thus, it is practical that the PPG sensor can be integrated with or attached to the housing of a pacemaker or Implantable Cardioverter-Defibrillator (ICD), as can be appreciated from FIGS. 7A and 8 as discussed below. Alternative locations for implantation of the PPG sensor include, but are not limited to, the patient's abdomen.

In accordance with an embodiment of the present invention, the plethysmography signal indicative of changes in arterial blood volume can be an impedance plethysmography (IPG) signal. An impedance plethysmography sensor can measure changes in blood volume for a specific body segment. As the arterial blood volume changes, the electrical resistance also changes. For certain embodiments it is preferred that a plurality of cardiac cycles of the obtained IPG signal are averaged to produce an IPG signal that is an averaged IPG signal.

The IPG sensor can be implanted, e.g., in the pectoral region of a patient. Thus, it is practical that the IPG sensor can be integrated with or attached to the housing or a pacemaker or ICD, as can be appreciated from FIGS. 7A and 8 as discussed below. Alternative locations for implantation of the IPG sensor include, but are not limited to, the patient's abdomen.

In still other embodiments, the plethysmography signal can be a signal output by a sensor including a piezo-electric diaphragm. Alternative sensors that can be used to produce the plethysmography signal, include, but are not limited to, a close range microphone, a sensor including a small mass on the end of a piezo bending beam with the mass located on the surface of a small artery, a transmission mode infrared motion sensor sensing across the surface of a small artery, or a micro-electro-mechanical systems (MEMS) accelerometer located on the surface of a small artery. Such alternative sensors can be located, e.g., on the tip of a short lead connected to a device that is subcutaneously implanted. The implanted sensor can be extra vascular, and a sufficient distance from the patient's heart such that meaningful changes in the amount of time it takes a pulse wave originating in the heart to reach the implanted sensor can be detected, thereby enabling changes in arterial blood pressure to be detected. For example, the implanted sensor (used to obtain the signal indicative of changes in arterial blood volume) can be at least 10 mm from the patient's aortic root. Such a sensor can be implanted, e.g., in the pectoral region of a patient. An alternative location for implantation of the sensor includes, but is not limited to, the patient's abdominal region.

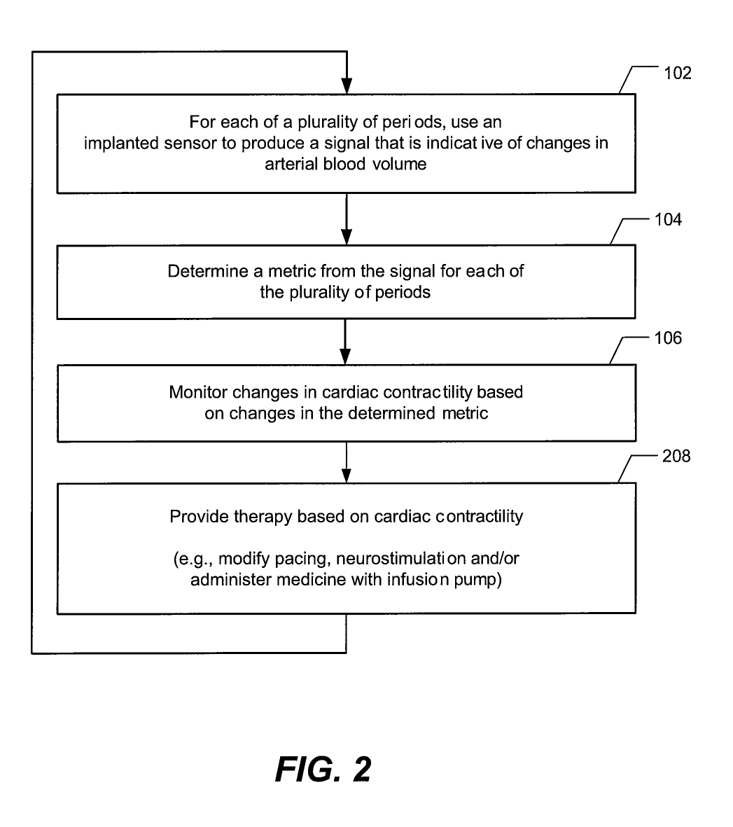

Referring now to FIG. 2, a flowchart of an embodiment is shown. Steps 102, 104 and 106 are the same as in FIG. 1, and thus need not be described again.

In step 208, therapy can be provided based on the determined cardiac contractility to improve the cardiac contractility. The determined cardiac contractility can also provide feedback to the therapy.

In one embodiment, at least one pacing parameter of a cardiac stimulation device can be adjusted to modify the cardiac contractility. Exemplary pacing parameters that can be changed to adjust the cardiac contractility include AV delay, V to V timing in a cardiac resynchronization therapy (CRT) device, as well as base rate, or electrode configuration and pace timing in a multi-electrode lead, but are not limited hereto.

A set of pacing parameters can provide higher or lower cardiac contractility. If there is a substantially lower cardiac contractility when certain pacing parameters (such as AV delay and/or V-V timing, or electrode configuration and pace timing in a multi-electrode lead) are adjusted, this may be because there is dyssynchrony. Similarly, if there is an increase in cardiac contractility when pacing is performed (compared to intrinsic rhythm where pacing is not performed), pacing may have reduced dyssynchrony (i.e., increased synchrony of myocardial contraction) or decreased the amount of mitral regurgitation.

In one embodiment, pacing parameter(s) can be adjusted based on the monitored cardiac contractility. For example, the pacing parameter(s) can be adjusted to modify the monitored cardiac contractility to be closer to a desired cardiac contractility. The desired cardiac contractility can be the maximal achievable contractility available with any choice of parameters, or it may be specified for a patient population or can be patient specific.

Beat-to-beat changes in the signal recorded by the implanted sensor can indicate changes in cardiac contractility. The morphology of the signal indicates the strength of the contractile force that resulted in the ejection of blood as a pressure pulsation into the arteries. The morphology also reflects other characteristics of the blood and the vessels as the pressure pulsation traverses to reach the sensor site, such as blood density, vessel compliance, length of path and bifurcations. These factors do not typically change from beat-to-beat. When pacing parameters are changed, the relevant metrics described above can indicate a change in cardiac contractility.

In an alternate embodiment, neurostimulation can be started and/or adjusted based on the determined cardiac contractility. For example, in one embodiment, in periods of low cardiac contractility, neurostimulation (such as spinal neurostimulation) can be increased. This increase can be an increase number of daily periods of stimulation, length of neurostimulation or intensity of neurostimulation. Such an increase in neurostimulation can result in a desirable increase in cardiac contractility.

In another alternate embodiment, medicine can be supplied based on a detected low cardiac contractility. For example, a medicine, such as Dobutamine, can be provided from an implanted medicine pump based on the detected low cardiac contractility.

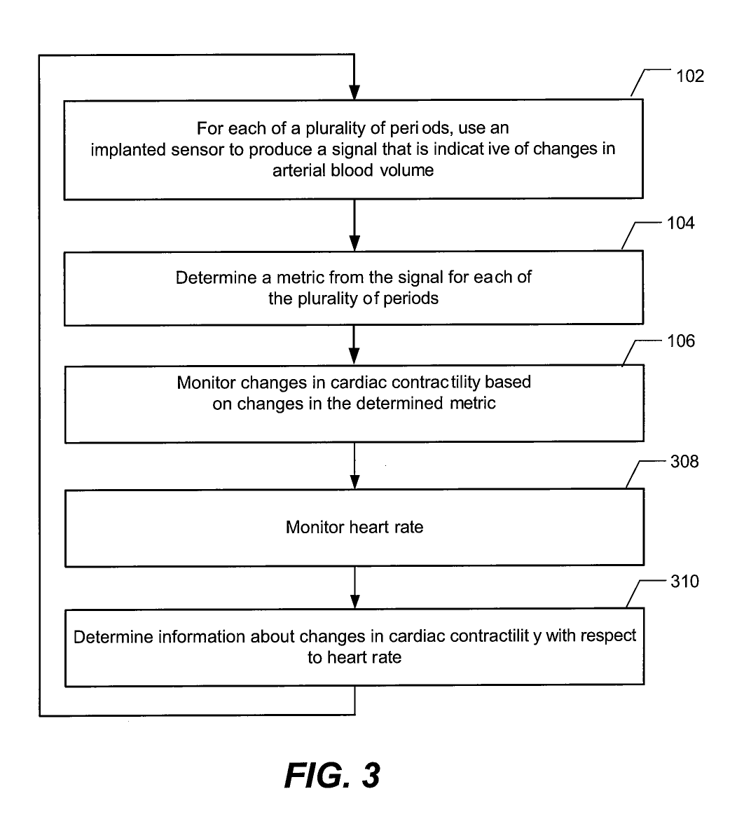

FIG. 3 shows an example where the heart's electrical activity is monitored along with the cardiac contractility. Steps 102, 104 and 106 are the same as in FIG. 1, and thus need not be described again. In step 308, the heart rate is monitored. This can be done by measuring the R-R interval from an IEGM or ECG, by measuring the period of plethysmography signal, or in some other fashion. In step 310, information about a change in cardiac contractility with respect to heart rate is determined.

For example, in response to increased oxygen demand, the heart rate will typically go up; if the cardiac contractility is poor during the period of elevated heart rate, then this can be determined. In this example, both the heart rate elevation and poor cardiac contractility can be determined. At that time, the pacing parameters can be adjusted to improve the cardiac contractility during the period of the elevated heart rate.

FIGS. 4-6 illustrate an exemplary signal waveform that is used to show various metrics indicative of the cardiac contractility. The signal waveform can be a PPG signal, an IPG signal, or some other plethysmography signal indicative of changes in arterial blood volume.

FIGS. 4-6 illustrate exaggerated examples with an increase in cardiac contractility in FIGS. 4B, 5B and 6B as compared to FIGS. 4A, 5A and 6A.

As shown in FIGS. 4A and 4B, the metric can be indicative of an upward slope of the signal indicative of changes in arterial blood volume, where the upward slope corresponds to the systolic portion of a cardiac cycle. The upward slope metric can be a maximum slope, average slope or some other metric indicative of upward slope. An increase in the upward slope metric is indicative of an increase in the cardiac contractility. Conversely, a decrease in the upward slope metric is indicative of a decrease in the cardiac contractility. No change in the upward slope metric is indicative of no change in the cardiac contractility.

As shown in FIGS. 5A and 5B, the metric can be indicative of a time between two features of a cycle of the signal indicative of changes in arterial blood volume. The two features can occur between and inclusive of a foot of the signal and a dicrotic notch.

Exemplary time metrics can include the time between the foot and the dicrotic notch, t₁; the time between the foot and max peak, t₂; the time between two different points on the upward slope such as the 25% and the 75% points, which are at 25% and 75% of the amplitude difference between the foot and peak, respectively, t₃; and the time between maximum upward slope and the maximum peak, t₄.

The Full Width Half Maximum (FWHM) value can also be used, but it may contain confounding information since the portion of the FWHM after the dicrotic notch is not related to the cardiac contractility. The metric may still provide information related to cardiac contractility if the portion of the FWHM after the dicrotic notch is relatively constant in response to changing pacing parameters.

A decrease in the time metric is indicative of an increase in the cardiac contractility. Conversely, an increase in the time metric is indicative of an decrease in the cardiac contractility. No change in the time metric is indicative of no change in the cardiac contractility.

As shown in FIGS. 6A and 6B, the metric can be indicative of signal amplitude where dicrotic notch occurs. This can be expressed in terms of a ratio, A/B, of the amplitude between the foot and the dicrotic notch, A, and the amplitude between the foot and the peak, B. An increase in this metric is indicative of an increase in the cardiac contractility. Conversely, a decrease in the metric is indicative of a decrease in the cardiac contractility. No change in the metric is indicative of no change in the cardiac contractility.

A metric can also be determined from a derivative of the signal. Specific features of the signal can be identified using a derivative of a signal. For example, zero crossings in the first derivative indicate peaks and valleys in the signal. Zero crossings in the second derivative indicate maximum or minimum slopes in the signal. Additional details about the first and second derivatives of the signal are provided below.

In one embodiment, a reduction of area under the curve of the first derivative can indicate low cardiac contractility.

Dyssynchrony can result in small “wiggles”, or slight changes in trajectory, or the signal indicative of changes in the arterial blood volume. The first derivative or a higher-order derivative may have zero crossings at the peaks and valleys of the “wiggles”. Thus, an increase in the number of zero crossings of the first derivative of the signal can be monitored to indicate an increase in dyssynchrony.

In one embodiment, the detection of a change in cardiac contractility, as a result of a change in pacing parameter, can be used to detect dyssynchrony of the heart's contractile force. Lack of capture by an implanted cardiac stimulation device can be detected based on the monitored cardiac contractility. Lack of capture can be determined when the cardiac contractility metric is the same as it is during intrinsic activity, rather than changing to values typical for during normal pacing.

Photoplethysmography (PPG) and impedance plethysmography (IPG) signals (collectively referred to as PPG/IPG signals), and other plethysmography signals, show changes in a patient's arterial system as a result of the patient's heart contracting, and such signals are indicative of changes in arterial blood volume. A PPG signal can be obtained using a PPG sensor, which can be an optical sensor including a light source and a light detector. An IPG signal can be obtained using an IPG sensor, which can include electrodes and circuitry used to measure the impedance between such electrodes. One or more such electrodes can be located on one or more leads, and/or a mechanical housing of an implanted device can act as one of the electrodes. Other types of plethysmography signals can be obtained using other types of sensors, as described above.

Monitoring of cardiac contractility can be improved if the PPG/IPG signals (or other plethysmography signals) used in the above described embodiments are appropriately processed. For example, recording of a plethysmography signal may be triggered, e.g., on an R wave, based on respiratory cycle, based on activity levels, etc. The plethysmography signal can be filtered to remove respiratory noise, motion artifacts, baseline drift, etc. For example, the signal can be band-pass filtered so that the pass-band is from about 0.7 to 10 Hz, although other pass bands can be used. Most of the respiration signal and high frequency noise can be removed by such filtering.

Additionally, an outlier removal process can be performed, to remove “bad” heart beats. For example, the outlier removal can be accomplished by grouping a plurality (e.g., 20) consecutive heart beats, determining a mean of the filtered plethysmography signal for the plurality of heart beats, and then comparing the determined mean to individual cycles of the filtered plethysmography signal. Further, outlier removal can be performed by removing each cardiac cycle of the filtered plethysmography signal that deviates by at least a threshold amount (e.g., 3 or some other number of standard deviations) from the mean of the plethysmography signal for the plurality of consecutive beats. The cycles of the plethysmography signal remaining after the outlier removal step can then be ensemble averaged, with the result being an average representation of the plethysmography signal for the plurality of consecutive beats, with noise and “bad” beats removed.

Thereafter, features of the plethysmography signal can be detected from the ensemble-averaged plethysmography signal and/or metrics can be determined from the ensemble-averaged plethysmography signal.

A first derivative of the ensemble-averaged plethysmography signal can be determined, and the location of the maximum positive slope of the ensemble-averaged plethysmography signal can be detected by determining the maximum of the first derivative. Further, since it is believed that the maximum positive slope cannot be more than 70% of an R-R interval away from an R-wave, if the location of the maximum positive slope is not within 70% of an R-R interval away from an R wave, a maximum positive slope detection can be determined to be bad, and not be used.

A second derivative of the ensemble averaged plethysmography signal can be determined to find local minimum and maximum. The locations of a maximum and a minimum are where the first derivative is equal to zero. The second derivative can be used to determine if a specific location is a maximum or a minimum. More specifically, if the second derivative is positive, then the point is at a minimum. If the second derivative is negative at a point, then the point is a maximum. The local minimum and local maximum that are closest to the maximum positive slope are the minimum and maximum amplitudes of the signal, which can be used, e.g., to determine the peak-to-peak amplitude of the ensemble averaged plethysmography signal. The maximum negative slope can be determined by identifying, from the first derivative, the local maximum that occurs after the maximum of the averaged plethysmography signal, but before the subsequent R-wave. From the second derivative, the dicrotic notch can be identified by identifying the local minimum following the maximum of the averaged plethysmography signal, but before the subsequent R-wave.

Alternative techniques for detecting features of and/or determining metrics from a plethysmography signal can be used, such as, but not limited to, techniques that rely on template matching, wavelets, neural networks, Fast Fourier Transform (FFT) and/or time warping. Alternatively, or additionally, techniques for detecting features of and/or metrics from a plethysmography signal can utilize respiratory cycles and R-R intervals.

Metrics of the plethysmography signal can also be determined based on the ensemble-averaged plethysmography signal. Such metrics can include, but are not limited to, area under the curve, Full Width Half Maximum (FWHM), and as already mentioned above, peak-to-peak amplitude time between features, upward slope and amplitude related metrics.

Typically, pacing capture will result in an increase in the cardiac contractility. If an increase in the cardiac contractility does not occur, this can be indicative that capture has not occurred.

### Exemplary Implantable System

FIGS. 7A-7C and 8 will now be used to describe exemplary implantable systems that can be used to monitor cardiac contractility, in accordance with embodiments of the present invention. Referring to FIG. 7A, the implantable system is shown as including an implantable stimulation device 710, which can be a pacing device and/or an implantable cardioverter defibrillator. The device 710 is shown as being in electrical communication with a patient's heart 712 by way of three leads, 720, 724 and 730, which can be suitable for delivering multi-chamber stimulation and shock therapy. The leads can also be used to obtain IEGM signals, for use in embodiments of the present invention. Instead of having leads with electrodes attached to the heart, it is also possible that subcutaneous electrodes can be used to obtain ECG signals. In still other embodiments, it is possible that the electrodes are located on the housing of the implantable device 710, and that such electrodes are used to obtain subcutaneous ECG signals. In this latter embodiment, the device 710 may not be capable of pacing and/or defibrillation, but rather, the implantable device 710 can be primarily for monitoring purposes.

The implantable system is also shown as including an implantable photoplethysmography (PPG) sensor 703 that can be used to produce a PPG signal, similar to the signals shown in FIGS. 4-6. Referring to FIGS. 7A, the PPG 703 sensor includes a light source 705 and a light detector 707. The light source 705 can include, e.g., at least one light-emitting diode (LED), incandescent lamp or laser diode. The light detector 707 can include, e.g., at least one photoresistor, photodiode, phototransistor, photodarlington or avalanche photodiode. Light detectors are often also referred to as photodetectors or photocells.

The light source 705 outputs light that is reflected or backscattered by surrounding patient tissue, and reflected/backscattered light is received by the light detector 707. In this manner, changes in reflected light intensity are detected by the light detector, which outputs a signal indicative of the changes in detected light. The output of the light detector can be filtered and amplified. The signal can also be converted to a digital signal using an analog to digital converter, if the PPG signal is to be analyzed in the digital domain. Additional details of exemplary implantable PPG sensors are disclosed in U.S. Pat. Nos. 6,409,675 and 6,491,639, both entitled “Extravascular Hemodynamic Sensor” (both Turcott), which are incorporated herein by reference.

A PPG sensor can use a single wavelength of light, or a broad spectrum of many wavelengths. In the alternate embodiments, the light source can be any source of radiant energy, including laser diode, heated filament, and ultrasound transducer. The detector can be any detector of radiant energy, including phototransistor, photodetector, ultrasound transducer, piezoelectric material, and thermoelectric material.

It is generally the output of the photodetector that is used to produce a PPG signal. However, there exist techniques where the output of the photodetector is maintained relatively constant by modulating the drive signal used to drive the light source, in which case the PPG signal is produced using the drive signal, as explained in U.S. Pat. No. 6,731,967, entitled” Methods and Devices for Vascular Plethysmography via Modulation of Source Intensity,” (Turcott), which is incorporated herein by reference.

The PPG sensor 703 can be attached to a housing 740 of an implantable device, which as mentioned above can be, e.g., a pacemaker and/or an implantable cardioverter-defibrillator (ICD), or a simple monitoring device. Exemplary details of how to attach a sensor module to an implantable cardiac stimulation device are described in U.S. patent application Ser. No. 10/913,942, entitled “Autonomous Sensor Modules for Patient Monitoring” (Turcott et al.), filed Aug. 4, 2004 (Attorney Docket No. A04P3019-US1), which is incorporated herein by reference. It is also possible that the PPG sensor 703 be integrally part of the implantable cardiac stimulation device 710. For example, the PPG sensor 703 can be located within the housing 740 of an ICD (or pacemaker) that has a window through which light can be transmitted and detected. In a specific embodiment, the PPG sensor 703 has a titanium frame with a light transparent quartz window that can be welded into a corresponding slot cut in the housing of the ICD. This will insure that the ICD enclosure with the welded PPG sensor will maintain a hermetic condition. In alternative embodiments, the PPG sensor can be remote from housing 740 and can communicate with components within the housing via a bus (e.g., including one or more wires), or wirelessly, but is not limited thereto.

Where the PPG sensor 703 is incorporated into or attached to a chronically implantable device 710, the light source 705 and the light detector 707 can be mounted adjacent to one another on the housing or header of the implantable device. The light source 705 and the light detector 707 are preferably placed on the side of the implantable device 710 that, following implantation, faces the chest wall, and are configured such that light cannot pass directly from the source to the detector. The placement on the side of the device 710 that faces the chest wall maximizes the signal to noise ratio by directing the signal toward the highly vascularized musculature, and shielding the source and detector from ambient light that enters the body through the skin. Alternatively, at the risk of increasing susceptibility to ambient light, the light source 705 and the light detector 707 can be placed on the face of the device 710 that faces the skin of the patient.

The implantable PPG sensor 703 outputs a PPG signal similar to signals shown in FIGS. 4-6. More specifically, the output of the light detector 705 can be an analog signal that resembles the signals in FIGS. 4-6. Such a signal can be filtered and/or amplified as appropriate, e.g., to remove respiratory affects on the signal, and the like. Additionally, the signal can be digitized using an analog to digital converter. Based on the PPG signal (and in some embodiments an ECG or IEGM obtained using implanted electrodes) metrics indicative of cardiac contractility, such as the upward slope, the time between two features of a cycle or relative amplitude, can be determined.

Still referring to FIG. 7A, to sense atrial cardiac signals and to provide right atrial chamber stimulation therapy, the device 710 is coupled to an implantable right atrial lead 720 having at least an atrial tip electrode 722, which typically is implanted in the patient's right atrial appendage. To sense left atrial and ventricular cardiac signals and to provide left-chamber pacing therapy, the device 710 is coupled to a “coronary sinus” lead 724 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, an exemplary coronary sinus lead 724 is designed to receive atrial and ventricular cardiac signals and to deliver left ventricular pacing therapy using at least a left ventricular tip electrode 726, left atrial pacing therapy using at least a left atrial ring electrode 727, and shocking therapy using at least a left atrial coil electrode 728.

The device 710 is also shown in electrical communication with the patient's heart 712 by way of an implantable right ventricular lead 730 having, in this embodiment, a right ventricular tip electrode 732, a right ventricular ring electrode 734, a right ventricular (RV) coil electrode 736, and an SVC coil electrode 738. Typically, the right ventricular lead 730 is transvenously inserted into the heart 712 so as to place the right ventricular tip electrode 732 in the right ventricular apex so that the RV coil electrode 736 will be positioned in the right ventricle and the SVC coil electrode 738 will be positioned in the superior vena cava. Accordingly, the right ventricular lead 730 is capable of receiving cardiac signals and delivering stimulation in the form of pacing and shock therapy to the right ventricle.

FIG. 7B illustrates an alternative embodiment of the implantable device 710. Here the housing 740 of the device is shown as small, thin, and oblong, with smooth surfaces and a physiologic contour which minimizes tissue trauma and inflammation. The oblong geometry of the housing 740 is desirable because it maximizes separation of electrodes 742 and prevents rotation of the monitor within the tissue pocket, thereby allowing interpretation of morphology features in an ECG sensed using electrodes 742. Two ECG electrodes 742 are shown, however more can be present. In the alternate embodiment illustrated in FIG. 7C, three ECG electrodes 742 are present, one at each apex of the triangle formed by the device housing 740. These three electrodes allow the three standard surfaces ECG leads I-III to be approximated. In an embodiment, four or more ECG electrodes might be used, with each orthogonal electrode pair providing orthogonal ECG signals. Alternatively, an embodiment lacking ECG electrodes is possible. A further alternative has a single ECG electrode with the monitor housing acting as the other electrode in the pair. U.S. Pat. No. 6,409,675, which was incorporated above by reference, provides some additional details of an implantable monitor that includes ECG electrodes on its housing and a PPG sensor. FIGS. 7B and 7C show that the implantable device 710 also includes a PPG sensor 703.

FIG. 8 will now be used to provide some exemplary details of the components of the implantable devices 710. The implantable device 710 can include logic to determine and monitor cardiac contractility.

Referring now to FIG. 8, each of the above implantable devices 710, and alternative versions thereof, can include a microcontroller 860. As is well known in the art, the microcontroller 860 typically includes a microprocessor, or equivalent control circuitry, and can further include RAM or ROM memory, logic and timing circuitry, state machine circuitry, and I/O circuitry. Typically, the microcontroller 860 includes the ability to process or monitor input signals (data) as controlled by a program code stored in a designated block of memory. The details of the design of the microcontroller 860 are not critical to the present invention. Rather, any suitable microcontroller 860 can be used to carry out the functions described herein. The use of microprocessor-based control circuits for performing timing and data analysis functions are well known in the art. In specific embodiments of the present invention, the microcontroller 860 performs some or all of the steps associated with monitoring cardiac contractility. Additionally, the microcontroller 860 may detect arrhythmias, and select and control delivery of anti-arrhythmia therapy.

Representative types of control circuitry that may be used with the invention include the microprocessor-based control system of U.S. Pat. No. 4,940,052, entitled “Microprocessor Controlled Rate-Responsive Pacemaker Having Automatic Rate Response Threshold Adjustment,” (Mann et. al.), and the state-machines of U.S. Pat. No. 4,712,555, entitled “Physiologically Responsive Pacemaker and Method of Adjusting the Pacing Interval Thereof,” (Thornander) and U.S. Pat. No. 4,944,298 entitled “Atrial Rate Based Programmable Pacemaker with Automatic Mode Switching Means,” (Sholder). For a more detailed description of the various timing intervals used within the pacing device and their inter-relationship, see U.S. Pat. No. 4,788,980 entitled “Pacemaker Having PVC Response and PMT Terminating Features,” (Mann et. al.). The '052, '555, '298 and '980 patents are incorporated herein by reference.

Depending on implementation, the device 710 can be capable of treating both fast and slow arrhythmias with stimulation therapy, including pacing, cardioversion and defibrillation stimulation. While a particular multi-chamber device is shown, this is for illustration purposes only, and one of skill in the art could readily duplicate, eliminate or disable the appropriate circuitry in any desired combination to provide a device capable of treating the appropriate chamber(s) with pacing, cardioversion and defibrillation stimulation. For example, where the implantable device is a monitor that does not provide any therapy, it is clear that many of the blocks shown may be eliminated.

The housing 740, shown schematically in FIG. 8, is often referred to as the “can”, “case” or “case electrode” and may be programmably selected to act as the return electrode for all “unipolar” modes. The housing 740 may further be used as a return electrode alone or in combination with one or more of the coil electrodes, 728, 736 and 738, for shocking purposes. The housing 740 can further include a connector (not shown) having a plurality of terminals, 842, 844, 846, 848, 852, 854, 856, and 858 (shown schematically and, for convenience, the names of the electrodes to which they are connected are shown next to the terminals). As such, to achieve right atrial sensing and pacing, the connector includes at least a right atrial tip terminal (A_{R} TIP) 842 adapted for connection to the atrial tip electrode 722.

To achieve left atrial and ventricular sensing, pacing and shocking, the connector includes at least a left ventricular tip terminal (V_{L} TIP) 844, a left atrial ring terminal (A_{L} RING) 846, and a left atrial shocking terminal (A_{L} COIL) 848, which are adapted for connection to the left ventricular ring electrode 726, the left atrial tip electrode 727, and the left atrial coil electrode 728, respectively.

To support right ventricle sensing, pacing and shocking, the connector further includes a right ventricular tip terminal (V_{R} TIP) 852, a right ventricular ring terminal (V_{R} RING) 854, a right ventricular shocking terminal (R_{V} COIL) 856, and an SVC shocking terminal (SVC COIL) 858, which are adapted for connection to the right ventricular tip electrode 732, right ventricular ring electrode 734, the RV coil electrode 726, and the SVC coil electrode 738, respectively.

An atrial pulse generator 870 and a ventricular pulse generator 872 generate pacing stimulation pulses for delivery by the right atrial lead 720, the right ventricular lead 730, and/or the coronary sinus lead 724 via an electrode configuration switch 874. It is understood that in order to provide stimulation therapy in each of the four chambers of the heart, the atrial and ventricular pulse generators, 870 and 872, may include dedicated, independent pulse generators, multiplexed pulse generators, or shared pulse generators. The pulse generators, 870 and 872, are controlled by the microcontroller 860 via appropriate control signals, 876 and 878, respectively, to trigger or inhibit the stimulation pulses.

The microcontroller 860 further includes timing control circuitry 879 which is used to control pacing parameters (e.g., the timing of stimulation pulses) as well as to keep track of the timing of refractory periods, noise detection windows, evoked response windows, alert intervals, marker channel timing, etc., which is well known in the art. Examples of pacing parameters include, but are not limited to, atrio-ventricular delay, interventricular delay and interatrial delay.

The switch bank 874 includes a plurality of switches for connecting the desired electrodes to the appropriate I/O circuits, thereby providing complete electrode programmability. Accordingly, the switch 874, in response to a control signal 880 from the microcontroller 860, determines the polarity of the stimulation pulses (e.g., unipolar, bipolar, etc.) by selectively closing the appropriate combination of switches (not shown) as is known in the art.

Atrial sensing circuits 882 and ventricular sensing circuits 884 may also be selectively coupled to the right atrial lead 720, coronary sinus lead 724, and the right ventricular lead 730, through the switch 874 for detecting the presence of cardiac activity in each of the four chambers of the heart. Accordingly, the atrial (ATR. SENSE) and ventricular (VTR. SENSE) sensing circuits, 882 and 884, may include dedicated sense amplifiers, multiplexed amplifiers, or shared amplifiers. The switch 874 determines the “sensing polarity” of the cardiac signal by selectively closing the appropriate switches, as is also known in the art. In this way, the clinician may program the sensing polarity independent of the stimulation polarity.

Each sensing circuit, 882 and 884, preferably employs one or more low power, precision amplifiers with programmable gain and/or automatic gain control, bandpass filtering, and a threshold detection circuit, as known in the art, to selectively sense the cardiac signal of interest. The automatic gain control enables the device 710 to deal effectively with the difficult problem of sensing the low amplitude signal characteristics of atrial or ventricular fibrillation. Such sensing circuits, 882 and 884, can be used to determine cardiac performance values used in the present invention. Alternatively, an automatic sensitivity control circuit may be used to effectively deal with signals of varying amplitude.

The outputs of the atrial and ventricular sensing circuits, 882 and 884, are connected to the microcontroller 860 which, in turn, are able to trigger or inhibit the atrial and ventricular pulse generators, 870 and 872, respectively, in a demand fashion in response to the absence or presence of cardiac activity, in the appropriate chambers of the heart. The sensing circuits, 882 and 884, in turn, receive control signals over signal lines, 886 and 888, from the microcontroller 860 for purposes of measuring cardiac performance at appropriate times, and for controlling the gain, threshold, polarization charge removal circuitry (not shown), and timing of any blocking circuitry (not shown) coupled to the inputs of the sensing circuits, 882 and 886.

For arrhythmia detection, the device 710 includes an arrhythmia detector 862 that utilizes the atrial and ventricular sensing circuits, 882 and 884, to sense cardiac signals to determine whether a rhythm is physiologic or pathologic. The timing intervals between sensed events (e.g., P-waves, R-waves, and depolarization signals associated with fibrillation) can be classified by the microcontroller 860 by comparing them to a predefined rate zone limit (i.e., bradycardia, normal, low rate VT, high rate VT, and fibrillation rate zones) and various other characteristics (e.g., sudden onset, stability, physiologic sensors, and morphology, etc.) in order to assist with determining the type of remedial therapy that is needed (e.g., bradycardia pacing, anti-tachycardia pacing, cardioversion shocks or defibrillation shocks, collectively referred to as “tiered therapy”). Additionally, the arrhythmia detector 862 can perform arrhythmia discrimination, e.g., using measures of arterial blood pressure determined in accordance with embodiments of the present invention. Exemplary details of such arrhythmia discrimination, including tachyarrhythmia classification, are discussed above. The arrhythmia detector 862 can be implemented within the microcontroller 860, as shown in FIG. 8. Thus, this detector 862 can be implemented by software, firmware, or combinations thereof. It is also possible that all, or portions, of the arrhythmia detector 862 can be implemented using hardware. Further, it is also possible that all, or portions, of the arrhythmia detector 862 can be implemented separate from the microcontroller 860.

In accordance with embodiments of the present invention, the implantable device 710 includes a cardiac contractility monitor 867, which can monitor cardiac contractility using the techniques described above with reference to FIGS. 1-6. The cardiac contractility monitor 867 can be implemented within the microcontroller 860, as shown in FIG. 8, and can be implemented by software, firmware, or combinations thereof. It is also possible that all, or portions, of the cardiac contractility monitor 867 to be implemented using hardware. Further, it is also possible that all, or portions, of the cardiac contractility monitor 867 to be implemented separate from the microcontroller 860.

The implantable devices 710 can be used to provide therapy based on the cardiac contractility determined by the cardiac contractility monitor 867. For example, the pacing can be provided through one or more of leads 842, 844, 846, 848, 852, 856 and 858 can be adjusted. Alternately, neurostimulation therapy provided through lead 859 can be started and/or adjusted. In a further embodiment, medication can be provided through medication pump 803 based on a determined low cardiac contractility.

The cardiac contractility monitor 867 can be used in a closed loop control system to detect changes in the cardiac contractility in response to changed pacing parameters or some other therapy so as to adjust the cardiac contractility.

The implantable device 710 can include a pacing controller 866, which can adjust a pacing rate and/or pacing intervals based on measures of arterial blood pressure, in accordance with embodiments of the present invention. The pacing controller 866 can be implemented within the microcontroller 860, as shown in FIG. 8. Thus, the pacing controller 866 can be implemented by software, firmware, or combinations thereof. It is also possible that all, or portions, of the pacing controller 866 can be implemented using hardware. Further, it is also possible that all, or portions, of the pacing controller 866 can be implemented separate from the microcontroller 860.

Still referring to FIG. 8, cardiac signals are also applied to the inputs of an analog-to-digital (A/D) data acquisition system 890. The data acquisition system 890 is configured to acquire IEGM and/or ECG signals, convert the raw analog data into a digital signal, and store the digital signals for later processing and/or telemetric transmission to an external device 802. The data acquisition system 890 can be coupled to the right atrial lead 720, the coronary sinus lead 724, and the right ventricular lead 730 through the switch 874 to sample cardiac signals across any pair of desired electrodes.

The data acquisition system 890 can be coupled to the microcontroller 860, or other detection circuitry, for detecting an evoked response from the heart 712 in response to an applied stimulus, thereby aiding in the detection of “capture”. Capture occurs when an electrical stimulus applied to the heart is of sufficient energy to depolarize the cardiac tissue, thereby causing the heart muscle to contract. The microcontroller 860 detects a depolarization signal during a window following a stimulation pulse, the presence of which indicates that capture has occurred. The microcontroller 860 enables capture detection by triggering the ventricular pulse generator 872 to generate a stimulation pulse, starting a capture detection window using the timing control circuitry 879 within the microcontroller 860, and enabling the data acquisition system 890 via control signal 892 to sample the cardiac signal that falls in the capture detection window and, based on the amplitude, determines if capture has occurred.

The implementation of capture detection circuitry and algorithms are well known. See for example, U.S. Pat. No. 4,729,376 entitled “Cardiac Pacer and Method Providing Means for Periodically Determining Capture Threshold and Adjusting Pulse Output Level Accordingly,” (Decote, Jr.); U.S. Pat. No. 4,708,142, entitled “Automatic Cardiac Capture Threshold Determination System and Method,” (Decote, Jr.); U.S. Pat. No. 4,686,988, entitled “Pacemaker System and Method for Measuring and Monitoring Cardiac Activity and for Determining and Maintaining Capture,” (Sholder); U.S. Pat. No. 4,969,467, entitled “Pacemaker with Improved Automatic Output Regulation,” (Callaghan et. al.); and U.S. Pat. No. 5,350,410, entitled “Autocapture System for Implantable Pulse Generator,” (Kleks et. al.), which patents are hereby incorporated herein by reference. The type of capture detection system used is not critical to the present invention.

The microcontroller 860 is further coupled to the memory 894 by a suitable data/address bus 896, wherein the programmable operating parameters used by the microcontroller 860 are stored and modified, as required, in order to customize the operation of the implantable device 710 to suit the needs of a particular patient. Such operating parameters define, for example, pacing pulse amplitude, pulse duration, electrode polarity, rate, sensitivity, automatic features, arrhythmia detection criteria, and the amplitude, wave shape and vector of each shocking pulse to be delivered to the patient's heart 712 within each respective tier of therapy. The memory 894 can also store data about cardiac contractility, as determined using embodiments of the present invention.

The operating parameters of the implantable device 710 may be non-invasively programmed into the memory 894 through a telemetry circuit 801 in telemetric communication with an external device 802, such as a programmer, transtelephonic transceiver, or a diagnostic system analyzer. The telemetry circuit 801 can be activated by the microcontroller 860 by a control signal 806. The telemetry circuit 801 advantageously allows intracardiac electrograms and status information relating to the operation of the device 710 (as contained in the microcontroller 860 or memory 894) to be sent to the external device 802 through an established communication link 804. The telemetry circuit can also be use to transmit data relating to cardiac contractility to the external device 802.

For examples of telemetry devices, see U.S. Pat. No. 4,809,697 entitled “Interactive Programming and Diagnostic System for use with Implantable Pacemaker” (Causey, III et al.); U.S. Pat. No. 4,944,299 entitled “High Speed Digital Telemetry System for Implantable Device” (Silvian); and U.S. Pat. No. 6,275,734 entitled “Efficient Generation of Sensing Signals in an Implantable Medical Device such as a Pacemaker or ICD” (McClure et al.), which patents are hereby incorporated herein by reference.

The implantable device 710 additionally includes a battery 811 which provides operating power to all of the circuits shown in FIG. 8. If the implantable device 710 also employs shocking therapy, the battery 811 should be capable of operating at low current drains for long periods of time, and then be capable of providing high-current pulses (for capacitor charging) when the patient requires a shock pulse. The battery 811 should also have a predictable discharge characteristic so that elective replacement time can be detected.

The implantable device 710 can also include a magnet detection circuitry (not shown), coupled to the microcontroller 860. It is the purpose of the magnet detection circuitry to detect when a magnet is placed over the implantable device 710, which magnet may be used by a clinician to perform various test functions of the implantable device 710 and/or to signal the microcontroller 860 that the external programmer 802 is in place to receive or transmit data to the microcontroller 860 through the telemetry circuits 801.

As further shown in FIG. 8, the device 710 is also shown as having an impedance measuring circuit 813 which is enabled by the microcontroller 860 via a control signal 814. The known uses for an impedance measuring circuit 813 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds and heart failure condition; detecting when the device has been implanted; measuring stroke volume; and detecting the opening of heart valves, etc. The impedance measuring circuit 813 is advantageously coupled to the switch 874 so that any desired electrode may be used. The impedance measuring circuit 813 can be used to obtain an impedance plethysmography (IPG) signal, which can be used in certain embodiments of the present invention to monitor cardiac contractility.

In the case where the implantable device 710 is also intended to operate as an implantable cardioverter/defibrillator (ICD) device, it should detect the occurrence of an arrhythmia, and automatically apply an appropriate electrical shock therapy to the heart aimed at terminating the detected arrhythmia. To this end, the microcontroller 860 further controls a shocking circuit 816 by way of a control signal 818. The shocking circuit 816 generates shocking pulses of low (up to 0.5 Joules), moderate (0.5-10 Joules), or high energy (11 to 40 Joules), as controlled by the microcontroller 860. Such shocking pulses are applied to the patient's heart 712 through at least two shocking electrodes, and as shown in this embodiment, selected from the left atrial coil electrode 728, the RV coil electrode 736, and/or the SVC coil electrode 738. As noted above, the housing 740 may act as an active electrode in combination with the RV electrode 736, or as part of a split electrical vector using the SVC coil electrode 738 or the left atrial coil electrode 728 (i.e., using the RV electrode as a common electrode).

The above described implantable device 710 was described as an exemplary pacing device. One or ordinary skill in the art would understand that embodiments of the present invention can be used with alternative types of implantable devices. Accordingly, embodiments of the present invention should not be limited to use only with the above described device.

The implantable sensor can also be part of a stand alone device that is not part of a pacing system. Such a stand alone device can monitor cardiac contractility and output indications of the cardiac contractility . . . .

The present invention has been described above with the aid of functional building blocks illustrating the performance of specified functions and relationships thereof. The boundaries of these functional building blocks have often been arbitrarily defined herein for the convenience of the description. Alternate boundaries can be defined so long as the specified functions and relationships thereof are appropriately performed. Any such alternate boundaries are thus within the scope and spirit of the claimed invention. For example, it would be possible to combine or separate some of the steps shown in the flow diagrams. Further, it may be possible to change the order of some of the steps shown in flow diagrams, without substantially changing the overall events and results. For another example, it is possible to change the boundaries of some of the blocks shown in FIG. 8.

The previous description of the preferred embodiments is provided to enable any person skilled in the art to make or use the embodiments of the present invention. While the invention has been particularly shown and described with reference to preferred embodiments thereof, it will be understood by those skilled in the art that various changes in form and details may be made therein without departing from the spirit and scope of the invention.

What is claimed is:

1. For use with an implanted system, a method for monitoring cardiac contractility, comprising:
(a) for each of a plurality of periods, using an implanted sensor to produce a signal that is indicative of changes in arterial blood volume;
(b) determining a metric from the signal for each of the plurality of periods; and
(c) monitoring changes in cardiac contractility based on changes in the determined metric.

2. The method of claim 1, wherein:
step (a) comprises using an implanted photoplethysmography (PPG) sensor to produce a photoplethysmography signal that is indicative of changes in arterial blood volume.

3. The method of claim 1, wherein:
step (a) comprises using an implanted impedance plethysmography sensor to produce an impedance plethysmography (IPG) signal that is indicative of changes in arterial blood volume.

4. The method of claim 1, wherein:
the metric determined at step (b) is indicative of an upward slope of the signal; and
detecting an increase in the metric at step (c) is indicative of an increase in the cardiac contractility; and
detecting a decrease in the metric of step (c) is indicative of a decrease of the cardiac contractility.

5. The method of claim 1, wherein:
the metric determined at step (b) is indicative of a time between two features of a cycle of the signal, the two features occurring between and inclusive of a foot indicative of beginning of systole and a dicrotic notch indicating closing of the aortic valve during the cycle of the signal;
detecting a decrease in the metric at step (c) is indicative of an increase in the cardiac contractility; and
detecting an increase in the metric at step (c) is indicative of a decrease in the cardiac contractility.

6. The method of claim 1, wherein:
the metric determined at step (b) is indicative of an amplitude of the signal where a dicrotic notch of the signal occurs;
detecting an increase in the metric at step (c) is indicative of an increase in the cardiac contractility; and
detecting a decrease in the metric at step (c) is indicative of a decrease of cardiac contractility.

7. The method of claim 1, wherein:
the metric determined at step (b) is determined from a derivative of the signal.

8. The method of claim 1, further comprising:
(d) adjusting at least one pacing parameter based on the monitored cardiac contractility.

9. The method of claim 8, wherein:
step (d) comprises adjusting the at least one pacing parameter to modify the monitored cardiac contractility be closer to a desired cardiac contractility.

10. The method of claim 1, further comprising:
(d) adjusting at least one pacing parameter and determining a change in the monitored cardiac contractility; and
(e) using the change in monitored cardiac contractility to determine a presence of dyssynchrony or a change in dyssynchrony.

11. The method of claim 1, further comprising:
(d) providing therapy based on the determined cardiac contractility.

12. An implantable system for monitoring cardiac contractility comprising:
an implantable sensor configured to produce a signal that is indicative of changes in arterial blood volume for each of a plurality of periods; and
a monitor configured to determine a metric from the signal for each of the plurality of periods; and
monitor changes in cardiac contractility based on changes in the metric.

13. The implantable system of claim 12, wherein:
the implantable sensor comprises an implantable photoplethysmography sensor configured to produce a photoplethysmography (PPG) signal that is indicative of changes in arterial blood volume.

14. The implantable system of claim 12, wherein:
the implantable sensor comprises an implantable impedance plethysmography sensor configured to produce an impedance plethysmography (IPG) signal that is indicative of changes in arterial blood volume.

15. The implantable system of claim 12, wherein:
the monitor is configured to determine the metric such that the metric is indicative of an upward slope of the signal;
detect an increase in the metric as indicative of an increase in the cardiac contractility; and
detect a decrease in the metric as indicative of a decrease in the cardiac contractility.

16. The implantable system of claim 12, wherein:
the monitor is configured to determine the metric such that the metric is indicative of a time between two features of a cycle of the signal, the two features occurring between and inclusive of a foot indicative of beginning of systole and a dicrotic notch indicative of the closing of the aortic valve during the cycle of the signal;
detect a decrease in the metric as indicative of an increase in the cardiac contractility; and
detect an increase in the metric as indicative of a decrease in cardiac contractility.

17. The implantable system of claim 12, wherein:
the monitor is configured to determine the metric such that the metric is indicative of a signal amplitude of where a dicrotic notch occurs expressed in terms of a fraction of a difference between a maximum and a minimum amplitude;
detect an increase in the metric as indicative of an increase in the cardiac contractility; and
detect a decrease in the metric as indicative of a decrease in the cardiac contractility.

18. The implantable system of claim 12, wherein:
the monitor is configured to determine the metric from a derivative of the signal.

19. The implantable system of claim 12, further comprising:
a pulse generator configured to produce cardiac pacing pulses; and
a pacing controller configured to control pacing parameters of the pacing pulses produced by the pulse generator;
wherein the pacing controller is configured to adjust at least one pacing parameter based on the monitored cardiac contractility.

20. The implantable system of claim 12, further comprising:
a component to provide therapy based on the determined cardiac contractility.

21. The implantable system of claim 12, further comprising:
a pulse generator configured to produce cardiac pacing pulses; and
wherein the monitor is configured to determine lack of capture by one or more of the pulses based on the monitored cardiac contractility.

22. For use with an implanted system, a method for monitoring cardiac contractility, comprising:
(a) using an implanted sensor to produce a plethysmography signal that is indicative of changes in arterial blood volume; and
(b) monitoring changes in cardiac contractility based on changes in the plethysmography signal.

23. The method of claim 22, further comprising determining a metric from the signal.

24. The method of claim 23, wherein step (b) includes monitoring changes in cardiac contractility based on changes in the determined metric.
